(19) United States
(12) Patent Application Publication
(10) Pub. No.: US 2024/0358471 A1
(43) Pub. Date: Oct. 31, 2024

(54) TISSUE MARKER WITH THERAPEUTIC PROPERTIES

(71) Applicant: Videra Surgical Inc., San Francisco, CA (US)

(72) Inventors: Alex Vayser, Mission Viejo, CA (US); Taylor Dean, Chicago, IL (US)

(21) Appl. No.: 18/643,613

(22) Filed: Apr. 23, 2024

Related U.S. Application Data
(60) Provisional application No. 63/461,768, filed on Apr. 25, 2023.

(51) Int. Cl. A61B 90/00; A61N 5/10

(52) U.S. Cl. CPC A61B 90/39; A61N 5/1001; A61N 5/1071; A61B 2090/392; A61B 2090/3966; A61B 2090/3991; A61B 2090/3995

(57) ABSTRACT

An implantable marker is configured to deliver a therapy to a patient and includes a marker and a target isotope. The marker is configured to enhance visibility of a treatment region of the patient when implanted in the patient and viewed with an imaging modality. The target isotope is coupled to at least a portion of the marker, and has a non-radioactive state and a radioactive state. At least a portion of the target isotope is configured to be induced from the non-radioactive state to the radioactive state when the target isotope is irradiated with one or more of gamma rays, neutrons, alpha particles, beta particles, or ions. The target isotope emits a localized therapeutic dose of radiation to target tissue adjacent the implantable marker when the target isotope is in the radioactive state.

### CROSS-REFERENCE TO RELATED PATENT DOCUMENTS

The present patent application is a non-provisional of, and claims the benefit of U.S. Provisional Patent Application No. 63/461,768 (Attorney Docket No. 5210.005PRV) filed on Apr. 25, 2023; the entire contents of which are incorporated herein by reference.

The present patent application is also related to U.S. patent application Ser. No. 18/166,393 (Attorney Docket No. 5210.002US3) filed on Feb. 8, 2023; the entire contents of which are incorporated herein by reference.

### BACKGROUND

Marking target tissue for treatment can present challenges since the markers must be visible under radiography, magnetic resonance imaging (MRI), ultrasound imaging, or other modalities used. In some circumstances it may be desirable to provide markers that are visible with one imaging modality while not visible with a second imaging modality. It may also be desirable to secure the markers to the target tissue or adjacent tissue, so the markers do not move. The marker may be used to deliver a therapeutic agent to the target tissue. For at least these as well as other reasons, it may be desirable to provide improved markers.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 shows an example of a stent that may be used to deliver a therapeutic agent.

FIG. 2 shows an example of a balloon catheter that may be used to deliver a therapeutic agent.

FIG. 3 shows an example of orthopedic implants coated with a therapeutic agent that may prevent infection.

FIG. 4 shows an example of a linear accelerator used to irradiate target tissue.

FIG. 5 shows an example of a catheter used to deliver radiation to target tissue.

FIG. 6A shows an example of brachytherapy used to treat target tissue.

FIG. 6B shows an example of brachytherapy seeds.

FIG. 7 shows an example of a radiopaque marker used to delineate a tumor bed.

FIGS. 8-9 show use of the marker in FIG. 7 to provide a three-dimensional target for external or internal radiation therapy.

FIG. 10 shows an example of a rigid marker.

FIG. 11 shows examples of various clips which may be used as markers.

FIG. 12 shows a room-bound source used to activate a marker post closure.

FIG. 13 shows an applicator for radioactive activation of a marker post closure.

FIG. 14 shows a radioactive source mounted on a needle or probe to activate the marker post closure.

FIG. 15 shows an example of radioactive source delivered with an endoscope or any flexible catheter to activate a marker post closure.

FIG. 16 shows pre-closure activation of a marker using a portable radioactive source.

FIG. 17 shows pre-closure activation of a marker using a room-bound source.

FIG. 18 shows a radioactive activation device for activation by means of photon disintegration.

FIG. 19 shows a flowchart of the steps used for a pre-activation dosimetry calculation for a particular setup.

FIG. 20 shows a computerized tomography (CT) image of a breast cancer patient with an overlay of the tumor bed.

FIG. 21 shows a CT image of a breast cancer patient with an overlay of the implanted marker.

FIG. 22 shows a CT image of a breast cancer patient with an overlay of the radioactive portion of the marker.

FIG. 23 shows a CT image of a breast cancer patient with an overlay of the deposited dose resulting from a source after it has been activated to provide radioactivity.

FIG. 24 shows a dose volume histogram of the dose deposited in a tumor bed for a particular setup.

FIG. 25 shows an example where the radioactive source used for activation is placed at different positions in order to generate a particular activation pattern.

### DETAILED DESCRIPTION

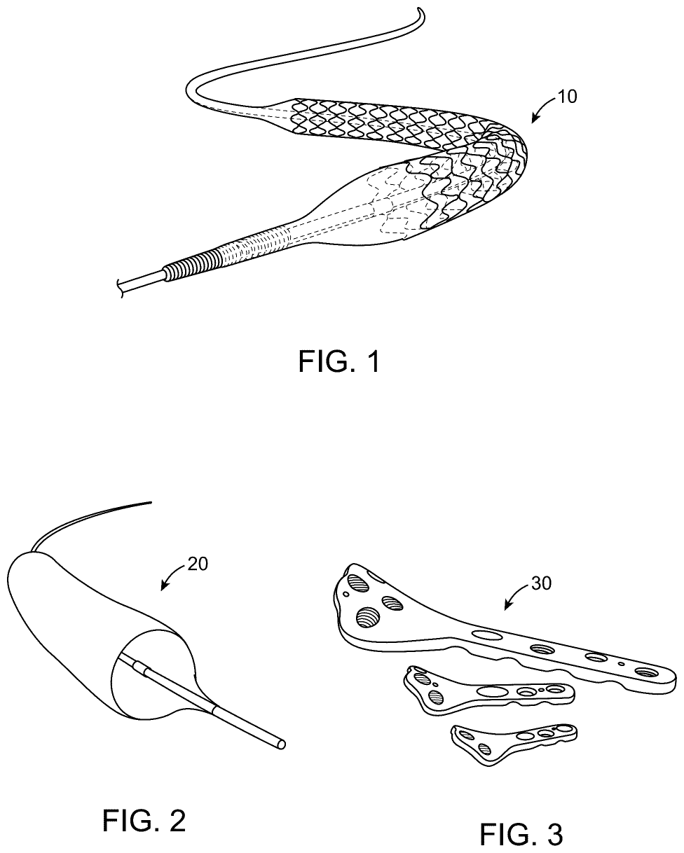

FIG. 1 illustrates a perspective view of a system for delivering surgical implants with integrated therapeutic properties that have been mostly limited to stents, balloons and orthopedic devices. In the early 2000s, the first vascular stent was implanted which was coated with a Sirolimus eluting drug to reduce restenosis in a vessel such as the device 10 seen in FIG. 1 which includes a stent coupled to a balloon catheter. Since then many devices have been commercialized, primarily focused to manage vascular disease and more specifically implanted in a lumen, like a vessel. In late 2011 mometasone furoate was used in conjunction with a bioabsorbable stent to reduce inflammation in a sinus.

Similar to stents, a balloon 20 was coated with antiproliferative coating (such as paclitaxel) to reduce restenosis of urethra as shown in FIG. 2.

Additionally, some orthopedic implants 30 have been coated with silver or other antimicrobial coatings to prevent infection and rejection of the implant as seen in FIG. 3.

Drug eluting stents and balloons have at least one thing in common. They are primarily used in applications inside a lumen or other hollow anatomical cavity which may be symmetric and these devices may reduce blockage or inflammation.

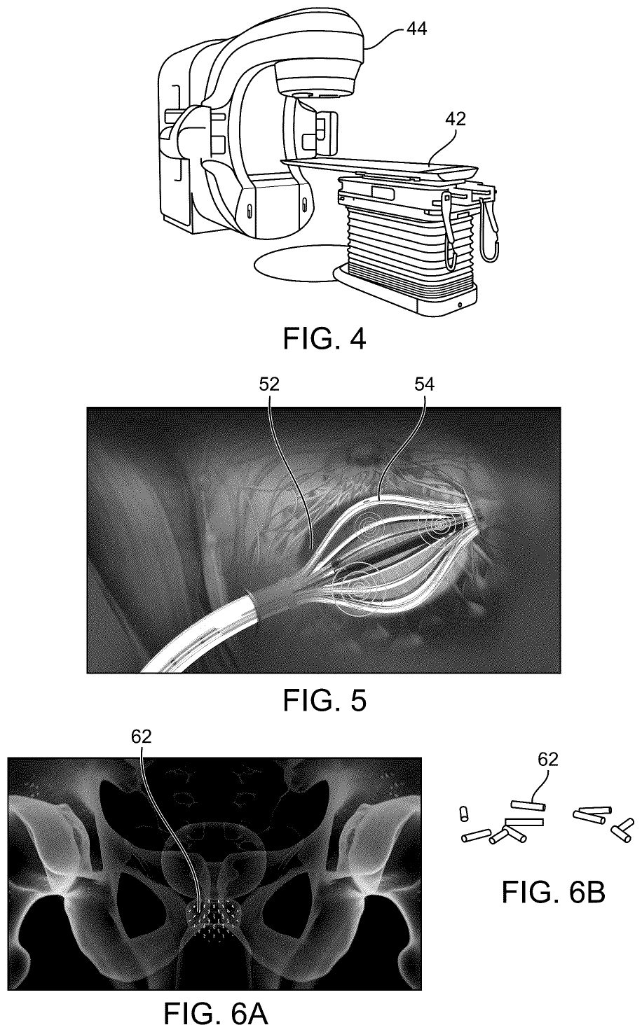

Currently, cancer patients who have undergone surgery to remove a malignant tumor are primarily treated with radiation, chemotherapy or both. Chemotherapy is a systematic treatment (whole body) and radiation covers an extended area, sometimes followed by an external higher energy boost to target a much smaller area where the original tumor was located. The radiation is typically delivered by a technology called Linear Accelerator (Linac). The patient may lie on a special bed 42 as shown in the FIG. 4 and the external radiation beam from the linear accelerator 44 is aimed at the target of treatment.

Although this is a current standard of care, the approach has limitations. Healthy tissue adjacent to the original tumor location is irradiated and in many situations, discoloration and potentially tissue death can occur. Also, depending on the diagnosis, the patient may have to come many times to the hospital to receive radiation treatment. Boost is delivered by the same equipment, however a much higher dose and in a much smaller area. The targeting for the boost is obviously critical.

There are some post-surgical therapies where an internal radioactive catheter is inserted into the cavity. As seen in FIG. 5, in the case of breast surgery and a post-surgical tumor bed 52, catheters 54 with a radiation source are inserted into the tumor bed. The patient is connected to the source and has to come in to to a treatment center such as a hospital or outpatient facility multiple times to receive treatment. After the completion of the treatment, the surgical opening is closed. The therapy is limited to patients with specific types of disease stage.

Aside from external radiation, solid tumors (without excision) have been treated with radioactive seeds (such as brachytherapy) seen as the white dots 62 in the x-ray image of the prostate in FIG. 6A. These seeds which are more clearly illustrated in FIG. 6B, generally contain a radioactive source such as Gold-198, Iridium-192, Iodine-125, Cesium-131. Since these seeds contain radioactive material, they are active and have to be handled with extreme caution. As they are emitting radiation, the surgeon and staff is exposed for the limited time they are moved from a shielded container into the patient. Many seeds are implanted into the area of interest and they are used to irradiate the area.

An interest is to develop a way to deliver therapy to the tumor bed post-surgical resection without having the patient having to be connected to an external device or come to a hospital or other treatment center multiple times for further treatment. The examples of devices and techniques disclosed herein may use an implant that may be deployed into tissue such as a solid tumor or a tumor bed during surgery, or deployed after surgery, and that may deliver therapeutic treatment to a localized region such as in the area of the malignancy. The therapeutic/drug delivery from this implant may last over a period of time after surgery.

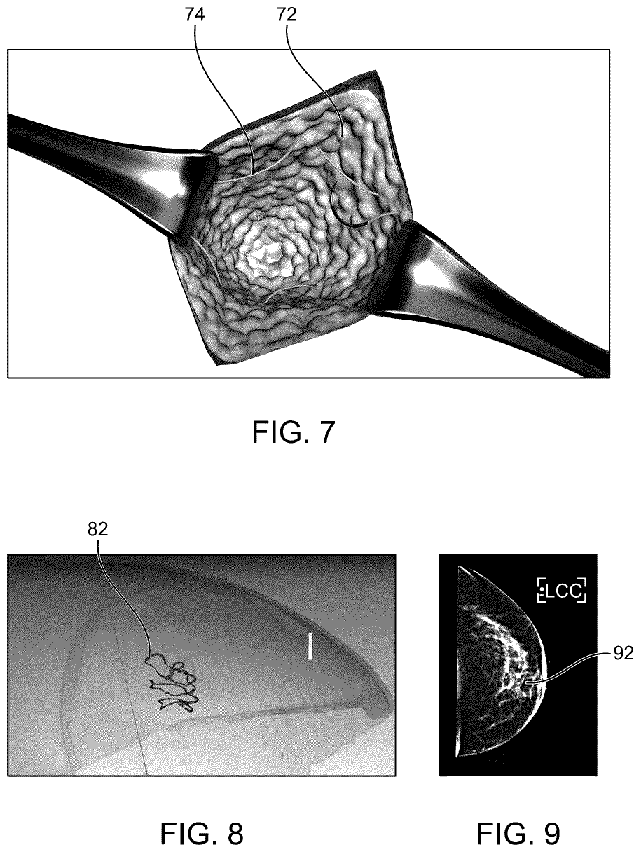

In previous patent applications such as U.S. patent application Ser. No. 18/166,393, previously incorporated herein by reference, a radiopaque tumor bed marker 74 that is deployed into the surgical tumor bed 72, as shown in FIG. 7 is disclosed. As described, the radiopaque tumor bed marker may be an elongated, flexible filament that is weaved into the perimeter of an open cavity after the tumor lesion has been excised. Since the properties of the filament are radiopaque and malleable, the contour and shape of the deployed marker accurately demarcates the tumor bed outline, thus providing a three-dimensional target for external or internal radiation therapy, which aids the radiation oncologist to develop a treatment plan 82, and 90, respectively, with contours in breast tissue as seen in FIGS. 8-9.

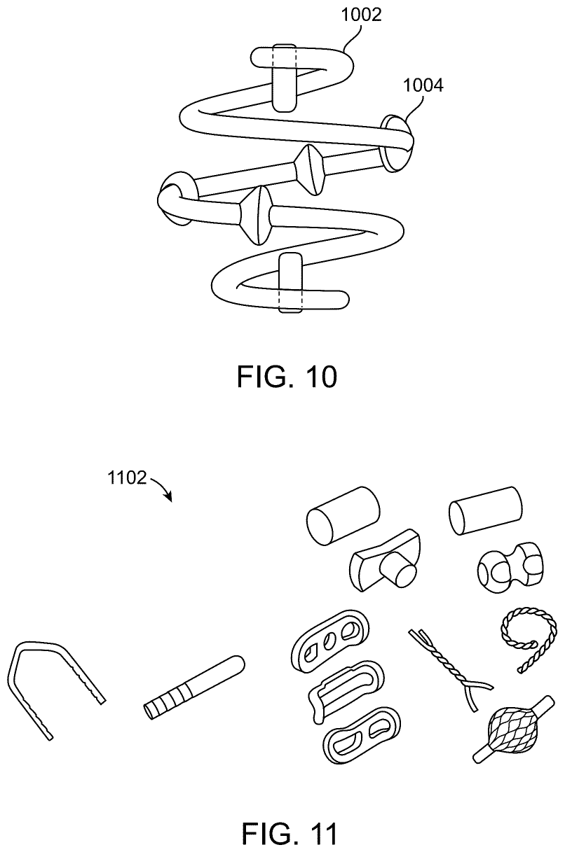

Examples disclosed herein may outline an additive applied to either the tumor bed marker material, such as a dopant or in form of external material such as a coating or microfibers to create a drug emitting tumor bed marker with therapeutic properties. The marker can be flexible or rigid. It can be permanent or can be absorbed over time. A rigid marker could be something similar to a Biozorb marker where the entire device 1002 has a drug emitting feature 1004 is shown in FIG. 10. In other examples, the therapeutic agent may be used to form all or a portion of the marker.

Individual clips 1102, such as hemostatic clips or biopsy clips can also be coated with therapeutic agent features as seen from FIG. 11, or formed therefrom.

The therapeutic properties are not limited to but may be characterized in one or more of the following categories:
1. Anti-microbial/anti-bacterial
2. Anti-inflammatory
3. Bleeding control/reduction/Hemostatic Agents
4. Serous Fluid Reduction
5. Estrogen Control/hormone receptor
6. Pain control/anesthetic
7. Radiation-radioactive particles/isotopes that are activated pre-closure (before the excision is closed) or post-closure (after the excision is closed).
8. Chemotherapy-internally or externally activated, such as by light, by paclitaxel, or with chemotherapy sensitizers.

The current state of the art has been primarily limited to application of coatings to a suture which is mostly used to close for example a surgical skin opening. A majority of the technology has been focused on anti-microbial properties. Surgical site infections present significant adverse effects clinically and economically. Primary areas of focus for antimicrobial coatings have been focused on bioactive agents or Active Pharmaceutical Ingredient (API) such as, but not limited to: Triclosan and Chlorhexidine applied to sutures (catgut, silk, nylon, polypropylene, PVDF (polyvinylidene fluoride), PTFE (polytetrafluoroethylene), PET (polyethylene terephthalate), PLA (polylactic acid), PCL (polycaprolactone), PGA (polyglycolide), PLGA poly(lactic-co-glycolic acid). PBAT (polybutylene adipate terephthalate), polybutesters, UHMWPE (ultra high molecular weight polyethylene), stainless steel, and other materials known in the art are examples of suture material.

Aside from Triclosan and Chlorhexidine, the following bioactive drugs can be used:
Chitosan, Totarol, Nitric oxide, Octenidine, Levofloxacin hydrochloride (LVFX-HCl), Tetracycline hydrochloride (TCH), Rifampicin, Octenidine dihydrochloride, Vancomycin Hydrochloride, Doxycycline

Beyond antimicrobial coatings, the summary below describes various categories of drug emitting coatings on the tumor bed markers which can be radiopaque, echogenic, or otherwise visible under an imaging modality.

### Pain Management/Local Anesthetic

Lidocaine, Bupivacaine such as Exparel which is a non-opioid, Xaracoll®, Marcaine, Posimir, Zynrelef. Other drugs may be applicable in this category.

### Anti-Inflammatory

Meloxicam, Ibuprofen, Aceclofenac, Dexamethasone, Tacrolimus, Busamide steroid and Toradol. Other analogs of Tacrolimus such as sirolimus, everolimus, biolimus, etc. may also be a useful therapeutic agent. Other drugs may be applicable in this category.

### Hemostatic Agents

Detran/Hyaloplasm acid (HA), Arista, Floseal.

### Serous Fluid Reduction

Microporous polysaccharide hemospheres.

### Radiopaque Coating (Increases Visibility During Radiography and CT Imaging)

Iodine micro-particles, gold and metallic (ZnO, TiO₂, Fe₂O₃, Cu, Cu₂O and MgO).

### Ferro or Superparamagnetic Coating (for Increased Visibility in MRI)

Holmium yttrium particles, Gadolinium (III) compounds, superparamagnetic iron oxides, superparamagnetic iron platinum particles or manganese (II) compounds.

A binding layer such as a hydrogel can be used with various drugs (polyglactin, poliglecprone, polydioxanone, polyglycolic acid, poliglecaprone. The hydrogel acts as a carrier agent for the API and its composition and properties can be manipulated for controlled drug release. There are multiple ways to deposit the API; internal to the core material, single layer with something like a hydrogel or multi-layer. Electrospinning is another way to deposit the API.

The current industry standard for applying a hydrogel coating to sutures, with or without an API, occurs post-manufacturing of the suture and utilizes a process called dip coating. Additional post-manufacturing coating practices include layer-by-layer coating, soaking, supercritical CO₂ assisted impregnation, and radiation grafting.

In dip coating the hydrogel polymers are first combined with a solvent and chosen API. The suture is pre-treated with a sanitizing agent then dipped in the hydrogel/API solution to apply the coating. Finally, the coated suture is subjected to a drying process to remove the solvent.

Layer-by-layer coatings apply sequential layers of polycations and polyanions, allowing the application of multiple variable and functional coating layers.

Soaking and supercritical CO₂ assisted impregnation allow the incorporation of APIs into the suture microfilaments. This impregnation is achieved using a solvent to swell the suture, allowing adsorption of the API. Additional coatings may be applied to the suture after API adsorption using one of the alternative coating methods listed.

Radiation grafting first irradiates a polymer suture and then a monomer is grafted to the suture and transformed to form chemical groups able to interact reversibly with a specific API.

Experimentally, APIs have been incorporated in absorbable polymer sutures during the suture fabrication process. These techniques include electrospinning (Blend, Emulsion, Coaxial, Side-by-side, Multiaxial) and Melt extrusion.

A radioactive version of the tumor bed marker is another example of this application. Any of the markers disclosed herein or disclosed in references incorporated herein may be modified to become a radioactive marker that can be used as described herein. As mentioned earlier, various APIs can be applied to the tumor bed marker or any type radiopaque or ferromagnetic or superparamagnetic marker (a marker will include tumor bed marker going forward), such as a filament implanted to deliver drug therapy to the site of interest, especially a tumor bed. One of the areas of interest mentioned earlier is a need to delivery radiation locally, from the “inside” of the cavity vs. from the outside. The opportunity is to create a marker and in this case a filament that is coated with or made out of stable isotope particles that are bound to the marker (we speak of “target isotope”), however are not radioactive yet, also referred to as “de-activated”, and are safe to handle (during production, storage, surgery, and after implantation). Although examples are directed toward deactivated isotopes in this description, one can include active isotopes as well, similar to (Iridium-192, Iodine-125, Palladium-103, Cobalt-60, Iodine-131, Yttrium-90, Holmium-166 Lutetium-177, Rhenium-186, Rhenium-188, Radium-223, Actinium-225, among many others). A challenge with active isotopes is their handling, however they can be included in this description. Once the marker is deployed, the user either (a) “activates” the marker, i.e., turns it radioactive (referred to herein as “activated isotope” or “radioactive isotope”), and closes the cavity (also referred to herein as pre-closure activation) or (b) activates it after the incision is closed-even several days up to months after implantation (also referred to herein as post-closure activation).

Radioactivity is a physical energy transfer process arising from unstable energy configurations within the nucleus of an atom. The most common reason is an unbalanced ratio between neutrons and protons. As a result, radioactivity can only be “activated” by nuclear reactions. The most common approaches for this are neutron activation and proton activation, followed by the less common ion activation. Another interesting activation option for examples of the radioactive marker disclosed herein may include photon disintegration.

In neutron activation, neutrons are directed at a target isotope with a high cross section and a stable but high ratio of neutrons to protons, thereby having a high likelihood to absorb neutrons and become unstable due to neutron excess. The cross section is given by the equivalent area that the nucleus poses to the incoming neutrons and is given in the unit Barn, which is 1E-24 cm²—wherein a high cross section is a cross section above 10 Barn, such as 100 Barn. A high ratio of neutrons to protons depends on the atomic number: for nuclei with atomic numbers in the range of 10, a high ratio would be above 1:1, while for nuclei with atomic numbers in the range of 70, a high ratio would be above 100:70. This approach is used for the generation of a vast majority of the radioisotopes used for medical and industrial applications. For example, highly pure and stable Lutetium-175 can be irradiated with neutrons to produce Lutetium-177 and small amounts of metastable Lutetium-177m after absorption of two neutrons. Lutetium-177 is a center isotope in radioligand therapy of prostate cancer using prostate-specific membrane antigen inhibitors. Other common radioisotopes used in medicine produced in a similar way are Cobalt-60, Palladium-103, Iodine-125, Iodine-131, Holmium-166, Rhenium-186, among many others.

In proton activation, ionized hydrogen atoms, i.e., protons, are accelerated commonly in cyclotrons and directed to target stable isotopes with a high cross-section which absorb the proton and turn unstable due to proton excess. An example includes Fluorine-18, which is produced by irradiating highly pure enriched stable Oxygen-18 atoms. The term enriched may be used to refer to a higher concentration of the stable isotope with respect with other stable isotopes of the same elements; Oxygen for example has three stable isotopes (Oxygen-16, Oxygen-17 and Oxygen-18) with an abundance of 99.75%, 0.03% and 0.22%; enriched Oxygen-18 would be a sample of Oxygen where the percentage of Oxygen-18 is far higher than the other stable isotopes, for example 80%. Fluorine-18 is the most used isotope for imaging in positron emission tomography (PET). In the particular case of Fluorine-18 activation, next to the proton absorption, a neutron is emitted. These types of changes in the nucleus of the target isotope are common and can be far more complex than a simple proton absorption. They do not only happen in proton activation, but also in neutron and ion activation, as well as in photon disintegration.

In ion activation bigger particles such as ionized Hydrogen-2, Hydrogen-3 or Helium-4 atoms are accelerated towards a target isotope. The resulting nuclear reactions are more complex, but tend to un-stabilize the target nuclei by creating a proton or neutron excess, where there is departure from the stable ratio of neutrons to protons as explained above. An example would be the absorption of a Helium-4 atom by the stable isotope Rubidium-87. This reaction results in the creation of an Yttrium-90 nucleus and the emission of a neutron. Yttrium-90 is a frequently used therapy isotope in nuclear medicine, for example used in selective internal radiotherapy (SIRT), also known as radio-embolization for the treatment of primary liver malignancies, as well as liver metastases of other primaries.

Finally, in the case of photon disintegration, the target isotope is irradiated with very high energy photons (X-rays or gamma rays), such as photons with energies above 500 keV, rather above 1 MeV, which results in neutrons, protons or combinations of neutrons and protons being emitted. If the target isotope had a low neutron to proton ratio (or low proton to neutron ratio respectively), it may turn radioactive. A low neutron to proton ratio—in contrast to the high neutron to proton ratio discussed above—for nuclei with atomic numbers in the range of 10 would be below 1:1, while for nuclei with atomic numbers in the range of 70, a low ratio would be below 100:70. An example would be irradiation of stable Palladium-104 with high-energy gamma rays resulting in the emission of a neutron from the nucleus. The resulting Palladium-103 is an isotope used commonly in brachytherapy seeds for the irradiation of prostate tissue such as in benign prostate hyperplasia or rather low-risk localized prostate cancer.

Note that none of the four activation processes is reversible. If activation took place, it cannot be stopped or slowed down, only accelerated.

In an example, we use all four activation paths described above to turn a target isotope embedded in the coating of the passive “de-activated” marker (or otherwise coupled or formed into the marker) into an active radioactive one. Alternatively, the target isotope can be radiopaque or superparamagnetic or ferromagnetic and can be the main material that forms the marker. A simplified formula for calculating the resulting radioactivity given the activation of a stable isotope is given by:

$$Activity after activation = Target isotope cross section * target isotope mass ( in the marker ) * Avogadro ’ s number * flux of neutrons * ( 1 - exp ( - 0.692 * half - life of resulting radioactive isotope * activation time ) ) \text{} / atomic weight of the target isotope$$

Ideally, but not limiting, the resulting radioactive marker emits very low energy photons, X-rays or gamma rays (<50 keV, like the ones emitted by Iodine-125 or Palladium-103), beta minus particles (<3 MeV, like the ones emitted by Copper-67, Lutetium-177, Yttrium-90, Iodine-131 or Terbium-161), Auger electrons (like Terbium-161) or alpha particles (<10 MeV, like the ones emitted by Bismuth-213, Radium-223 or Actinium-225, or from the short half-life daughter of Lead-212, Bismuth-212). The range of the low energy gamma emissions or beta and alpha particles may be selected to enable depositing a desired dose to the tumor bed and the presence of radiosensitive structures in the direct vicinity. For example, if the tumor bed thickness to be irradiated is 1 cm, then isotopes with a range above 1 cm should be selected. Yttrium-90, Holmium-166, or Rhenium-188 all have beta particle ranges of roughly 2 cm in soft tissue, so they may be good alternatives. In contrast, if only an irradiation of only immediate tissue next to the marker is desired, for example, since a particular closely located organ should not get a high dose, an isotope can be selected with emissions with low penetration. For example, Lutetium-177 beta particles only penetrate roughly 2 mm in tissue. Also alpha particles of Actinium-225 are a good short range option with less than 100 μm.

Target isotopes that get activated by neutrons can be seen in Table 1 below. While this list is not exhaustive, it does contain examples of target isotopes that are available and radioactive isotopes that are useful for the application of the techniques described herein.

In Table 1 isotopes are abbreviated in their conventional way. The nuclear reactions are described using conventional nomenclature, where N is the radioactive isotope element. For example, ^{z-1}Nₐ(n,gamma)^{z}Nₐ means that a nucleus of element N with a mass number “z-1” and an atomic number “a” is irradiated with neutrons n, resulting in gamma rays being emitted and a nucleus absorbing a neutron which yields the production of a nucleus with mass number “z” and the same atomic number “a”. The following definitions are used: z is the mass number of the radioactive isotope, a is its atomic number, n stands for neutron, d for deuterium nucleus, and p for proton.

TABLE 1

| Radioactive isotope | Target isotope | |
|---|---|---|
| (resulting isotope | (isotope to be | Nuclear reaction |
| after activation) | activated) | (activation reaction) |
| Y-90 | Y-89 | ^{z-1}Nₐ(n, gamma)^{z}Nₐ |
| | Zr-91 | ^{z+1}Nₐ + 1(n, d) ^{z}Nₐ |
| Pd-103 | Pd-102 | ^{z-1}Nₐ(n, gamma) ^{z}Nₐ |
| | Rh-103 | ^{z}Nₐ₊₁(n, p) ^{z}Nₐ |
| | Pd-104 | ^{z+1}Nₐ(n, 2n) ^{z}Nₐ |
| I-125 | Te-125 | ^{z}Nₐ₊₁(n, p) ^{z}Nₐ |
| | Xe-126 | ^{z+1}Nₐ₊₁(n, d) ^{z}Nₐ |
| I-131 | Xe-132 | ^{z+1}Nₐ₊₁(n, d) ^{z}Nₐ |
| Ho-166 | Ho-165 | ^{z-1}Nₐ(n, gamma) ^{z}Nₐ |
| | Er-167 | ^{z+1}Nₐ₊₁(n, d) ^{z}Nₐ |
| Lu-177 | Lu-176 | ^{z-1}Nₐ(n, gamma) ^{z}Nₐ |
| | Hf-178 | ^{z+1}Nₐ₊₁(n, d) ^{z}Nₐ |
| Re-186 | Re-185 | ^{z-1}Nₐ(n, gamma) ^{z}Nₐ |
| | W-186 | ^{z}Nₐ₊₁(n, p) ^{z}Nₐ |
| | Os-187 | ^{z+1}Nₐ₊₁(n, d) ^{z}Nₐ |
| | Re-187 | ^{z+1}Nₐ(n, 2n) ^{z}Nₐ |
| Re-188 | Re-187 | ^{z-1}Nₐ(n, gamma) ^{z}Nₐ |
| | Os-189 | ^{z+1}Nₐ₊₁(n, d) ^{z}Nₐ |

Table 2 shows radioactive isotopes and their parent target isotopes that can be activated by alpha particles. Table 2 uses the same nomenclature as in Table 1. Additionally. He stands for a helium nucleus, and t for tritium.

TABLE 2

| Radioactive isotope | Target isotope | Nuclear reaction |
|---|---|---|
| Y-90 | Rb-87 | ^{z-3}Nₐ₋₂(He, n) ^{z}Nₐ |
| | Sr-88 | ^{z-2}Nₐ₋₁(He, d) ^{z}Nₐ |
| Pd-103 | Ru-99 | ^{z-4}Nₐ₋₂(He, gamma) ^{z}Nₐ |
| | Ru-100 | ^{z-3}Nₐ₋₂(He, n) ^{z}Nₐ |
| | Ru-101 | ^{z-2}Nₐ₋₂(He, 2n) ^{z}Nₐ |
| I-125 | Sb-121 | ^{z-4}Nₐ₋₂(He, gamma) ^{z}Nₐ |
| | Te-122 | ^{z-3}Nₐ₋₁(He, p) ^{z}Nₐ |
| | Te-123 | ^{z-2}Nₐ₋₁(He, d) ^{z}Nₐ |
| | Te-124 | ^{z-1}Nₐ₋₁(He, t) ^{z}Nₐ |
| | Sb-123 | ^{z-2}Nₐ₋₂(He, 2n) ^{z}Nₐ |
| I-131 | Te-130 | ^{z-1}Nₐ₋₁(He, t) ^{z}Nₐ |
| Ho-166 | Dy-164 | ^{z-2}Nₐ₋₁(He, d) ^{z}Nₐ |
| Lu-177 | Yb-176 | ^{z-1}Nₐ₋₁(He, t) ^{z}Nₐ |
| Re-186 | W-184 | ^{z-2}Nₐ₋₁(He, d) ^{z}Nₐ |
| Re-188 | W-186 | ^{z-2}Nₐ₋₁(He, d) ^{z}Nₐ |

Table 3 shows radioactive isotopes and their parent target isotopes that can be activated by high-energy photons. Table 3 uses the same nomenclature as used as in Table 1. Additionally. He stands for a helium nucleus, and t for tritium.

TABLE 3

| Radioactive isotope | Target isotope | Nuclear reaction |
|---|---|---|
| Y-90 | Zr-91 | ^{z+1}Nₐ₊₁(gamma, p) ^{z}Nₐ |
| | Zr-92 | ^{z+2}Nₐ₊₁(gamma, d) ^{z}Nₐ |
| Pd-103 | Pd-104 | ^{z+1}Nₐ(gamma, n) ^{z}Nₐ |
| | Pd-105 | ^{z+2}Nₐ(gamma, 2n) ^{z}Nₐ |
| I-125 | Xe-126 | ^{z+1}Nₐ₊₁(gamma, p) ^{z}Nₐ |
| | I-127 | ^{z+2}Nₐ(gamma, 2n) ^{z}Nₐ |
| I-131 | Xe-132 | ^{z+4}Nₐ₊₂(gamma, He) ^{z}Nₐ |
| Ho-166 | Er-167 | ^{z+1}Nₐ₊₁(gamma, p) ^{z}Nₐ |
| | Er-168 | ^{z+2}Nₐ₊₁(gamma, d) ^{z}Nₐ |
| Lu-177 | Hf-178 | ^{z+1}Nₐ₊₁(gamma, p) ^{z}Nₐ |
| | Hf-179 | ^{z+2}Nₐ₊₁(gamma, d) ^{z}Nₐ |
| | Ta-181 | ^{z+4}Nₐ₊₂(gamma, He) ^{z}Nₐ |
| Re-186 | Re-187 | ^{z+1}Nₐ(gamma, n) ^{z}Nₐ |
| | Os-187 | ^{z+1}Nₐ₊₁(gamma, p) ^{z}Nₐ |
| | Os-188 | ^{z+2}Nₐ₊₁(gamma, d) ^{z}Nₐ |
| Re-188 | Os-189 | ^{z+1}Nₐ₊₁(gamma, p) ^{z}Nₐ |
| | Os-190 | ^{z+2}Nₐ₊₁(gamma, d) ^{z}Nₐ |

An additional aspect of the examples disclosed herein is that the marker can be designed such that different target isotopes are used at different parts of the marker. This makes it possible to have areas for example of the tumor bed being irradiated generously where the marker has isotopes with emissions of high penetration like Yttrium-90, while other parts of the tumor bed are only irradiated in close proximity to the marker (for example, less than 3 mm), where the marker has isotopes with emissions of low penetration such as Lutetium-177. In this specification, high penetration refers to radiation penetration in human tissue above 1 cm, while low penetration refers to radiation penetration in human tissue below 3 mm. Such a flexibility can be of paramount importance if the marker is placed across heterogeneous tissue or in proximity to delicate structures, such that the final irradiation can be modulated by placing markers with different coating or having different radioactive portions, at different parts appropriately. So, the marker can consist of variety of stable isotopes on the same marker or one can offer single markers with specific stable isotopes and the user can chose which marker to use depending on the location of placement. One can imagine a kit of markers each with a different stable isotope. Or a series of markers with multiple stable isotopes along different portions of the marker.

It was mentioned above that beta-emitting or alpha-emitting isotopes can be selected as activated isotopes. Alpha-particles and beta-particles have a maximum penetration range in tissue. Beta minus particles with less than 3 MeV normally do not penetrate more than 2 cm in soft tissue. As for alpha particles with less than 50 MeV penetration in soft tissue is never above 1 mm. Yet, it also disclosed that the isotope can emit very low energy photons (i.e., below 50 keV) as in the case of iodine-125. Gamma rays do not have a penetration yet, if their energy is low due to the steep fall in absorption a practical maximum penetration range can be achieved.

The resulting marker (post activation) may or may not involve high energy gamma emissions (>60 keV, like the ones emitted by Technetium-99m or Indium-111, typical single-photon emission tomography, SPECT, isotopes) or beta plus/positron emissions with a short range (<10 mm, like the ones emitted by Fluorine-18 or Gallium-68, typical PET isotopes) if imaging their position in space using nuclear medicine imaging devices (such as SPECT, PET or gamma cameras) is desired. Such imaging may make sense to calculate the real applied dose on the tumor margin after implantation of the marker. Yet, if beta- or alpha-emissions are used, the resulting radioactive distribution may also be imaged detecting the Bremstrahlung beta and alpha particles emit in tissue with a gamma camera or a SPECT.

The resulting marker should have a radioactive half-life of several days to weeks (such as the half-life of Palladium-103, Iodine-125 or Iodine-131) in order to deliver a relevant dose for the surrounding tissue over a longer period. This makes radiation protection considerations less relevant than short-lived isotopes which require high dose rates to deliver the same dose that a long-lived isotope delivers over a long period.

If emissions for imaging are desired, the half-life can be selected to be shorter to avoid irradiation of medical staff, family members and neighbors. For that means, the half-life should be ideally not more than 2 days.

To be able to activate the target isotope in the marker in vivo, i.e., after implanting, there are several alternatives possible. On the one hand, post site closure activation may occur when the marker is not radioactive during implantation, but it can be activated/made radioactive once a post closure irradiation of it takes place. On the other hand, pre-closure activation may occur when after placement in the tumor bed, activation means such as any of those disclosed herein, are brought into the wound or very close to it in order to turn the marker into a radioactive marker during the surgical intervention.

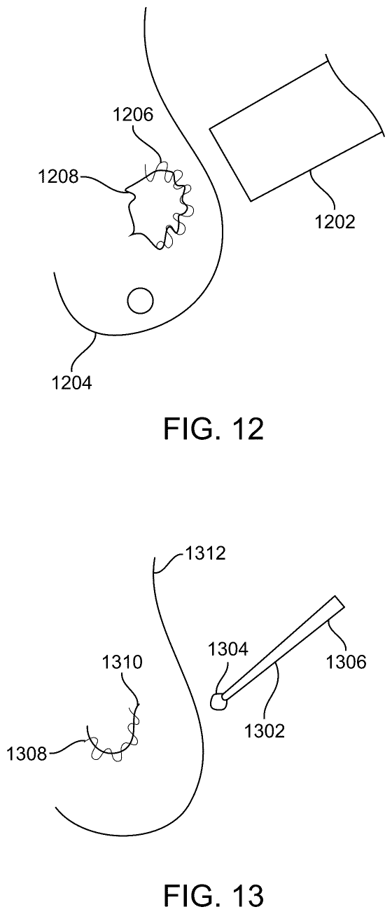

One option for post-closure activation is the use of a room-bound neutron beam directed to the marker as illustrated in FIG. 12. A patient 1204 (in this example a breast) has been implanted with a marker 1206 in the tumor bed 1208. A room-bound neutron beam emitted by a neutron source 1202 is directed to the marker 1206. Such an approach is used in neutron capture therapy, yet the way the target isotope is placed on the body is completely different. In neutron capture therapy, commonly a boron-modified amino acid-analogue (e.g., boronophenylalanine) is given to the patient intravenously. Cancer cells accumulate it more than healthy tissue, commonly due to the increased metabolic activity resulting in the increased uptake of amino acids. In our setup, the target isotope, which can be a mixture of stable Boron-10 and stable Boron-11, is part of the marker.

A portable radioactive source emitting neutrons can also be used to activate the marker 1308 in a tumor bed cavity 1310 in a patient 1312 which in this example is a breast, post closure percutaneously as illustrated in FIG. 13. The situation is similar to FIG. 12, yet the neutron source is replaced by a radioactive source holder 1302 comprising a radioactive source 1304 and a handle 1306.

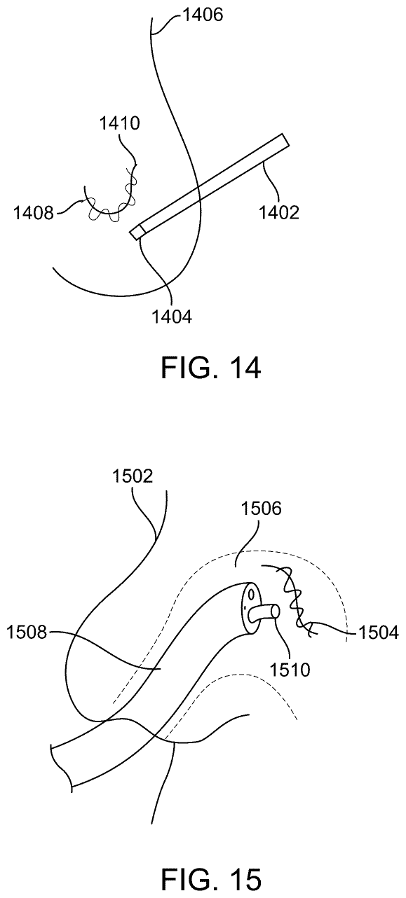

Yet another example of an activator for post-suture activation with a portable radioactive source is shown in FIG. 14. Here, a needle or other tissue piercing probe 1402 with an embedded radioactive source on its tip 1404 is inserted in the patient body 1406 (here, a breast) in close proximity to the marker 1408 such that its neutron emissions can activate the marker in the tissue cavity 1410.

Yet another example of an activator for post closure activation with a portable radioactive source is depicted in FIG. 15. Here, a patient 1502 (in this example, a patient's nose) has been implanted with a marker 1504 inside a cavity 1506 (the cavity could be due to tissue excision or it could be a natural opening like sinus or colon/rectum). An endoscope 1508 is used to introduce a flexible cannula with a radioactive source 1510 on its tip through a natural orifice (in this example a nostril) or through a surgical wound. The radioactive source is placed in close proximity of the marker.

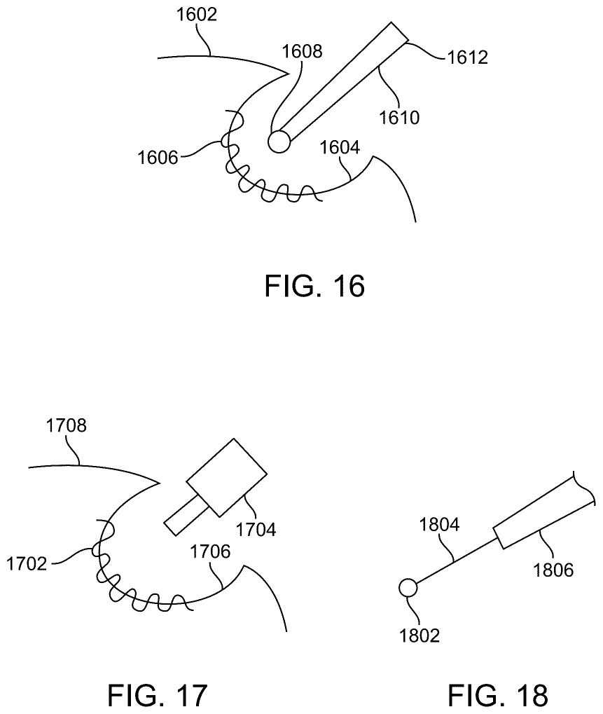

A pre-closure alternative using neutron activation is placing a portable neutron source inside the wound before closing it as illustrated in FIG. 16. Here, a patient 1602 with an open cavity 1604 has been implanted with a marker 1606. A radioactive source 1608 can be then put in the wound using a holder 1610 with a handle 1612 to activate the marker.

FIG. 17 shows an alternative example of the example seen in FIG. 16, where instead of a radioactive source, a room-bound beam emitter 1704 is used to activate the marker 1702 coupled to tissue in the cavity 1706 in patient 1708.

FIG. 18 shows an example used for the generation of high-energy photons for activation by means of photodisintegration. As mentioned above, such photons have energy above 500 keV, and ideally above 1 MeV. The photon source 1802 may be on the tip of elongate probe 1804 with a handle 1806.

Different neutron sources can be used, such as the combination of Beryllium-9 and Antimonium-124 (also known as AmBe), or Californium-252. Such neutron sources are radioactive materials or a combination of radioactive isotopes that result in a net spontaneous emission of neutrons.

If proton activation is the selected path, the patient can be irradiated with a room-bound proton beam directed to the tumor bed. This can be done by modifying a cyclotron such that the tumor bed can be irradiated, or alternatively using a proton therapy device which already can orient a proton beam towards a target area. The difference with proton therapy is that the protons are not meant to treat the tumor bed, but to activate the target isotope in the marker which in turns becomes radioactive and irradiates and treats the target tissue in the tumor bed or adjacent tissue. The Bragg peak of the proton therapy device may ideally be selected to be at the depth the marker is placed.

Like in the case of the neutron activation option, a proton source can also be placed in the wound pre-closure to activate the target isotope in the marker as illustrated in FIG. 16 or 17, or post closure as illustrated in (FIGS. 12, 13, 14 and FIG. 15 showing various configurations). Proton sources are rare but exist, like in the case of Lutetium-151 or Thalium-147. Such proton sources are radioactive materials or a combination of radioactive isotopes that result in a net spontaneous emission of protons.

In analogue to proton activation, ion activation of the target isotope in the marker can be achieved using ion therapy devices (room-bound ion activation) or using an ion source placed inside the body before closing the wound or close to the skin after implantation. Such a source can be an alpha emitter (e.g., Thorium-222, Radium-223, Actinium-225, Uranium-235, Uranium-238). Such ion sources are radioactive materials or a combination of radioactive isotopes that result in a net spontaneous emission of ions.

For photodisintegration also two options arise. High-energy photons (X-rays or gamma rays) can be applied to the tumor bed from outside using room-bound devices (e.g., Megavolt Linacs, or irradiation setups using radioactive isotopes like Cobalt-60 respectively), or alternatively a portable photon (X-ray or gamma ray) source can be placed in the tumor bed before closing and removed afterwards. Putting a source close to the skin after marker implantation is also possible. For the portable option, due to the high energies required for photodisintegration (at least 500 keV, ideally over 1 MeV), using radioactive isotopes as gamma source or using Megavolt Linacs irradiating an x-ray target inside of the patient or close to his/her skin.

A relevant aspect of the examples disclosed herein is the fact that the activation process, either if pre-closure or post-closure, either with a room-bound source or with a portable one, can be modulated, such that the degree of activation of different parts of the marker can be changed selectively. The activation source (independently if it is a room-bound beam or a portable radioactive source placed close to the patient) can be placed at different positions and with different orientations with respect to the patient. The different positions and orientations can be held for different time intervals. As a result, different parts of the marker will get activated differently and the resulting dose distribution of radiation from the marker to the target tissue can be modulated. FIG. 25 presents such a scenario.

In FIG. 25, a portable radioactive source 2508 on an elongate shaft or handle 2510 is placed first in position A and left there for a time t_A, and then placed in position B for t_B. By doing this, a different pattern of activation can be achieved than if the portable radioactive source is in position A for t_A+t_B. Positions A and B allow the radioactive source 2508 to irradiate and activate the marker 2506 disposed in cavity 2504 in patient 2502, here a patient's breast. The marker may here or any marker disclosed in this specification may be any of the examples of markers disclosed in this specification.

The above-mentioned method requires precise planning since any of the four proposed activation processes do not necessarily only activate the target isotope on the marker, but could also activate parts of the marker beyond the stable isotope, as well as biological tissue. Additionally, depending on the type of activation selected non-activation interactions between the activating means and biological tissue can occur.

Examples of undesired radiation burden due to the activation process may include any of the following examples.

Neutrons used to activate highly pure (i.e., 99.99% or higher) and enriched Rhenium-185 can also activate traces of Rhenium-187, as well as atoms of patient tissues in the surrounding areas of the tumor bed or adjacent tissue, such as Carbon-12, Hydrogen-1, Oxygen-16, etc. Note that with enriched we refer to the increase to the proportion of a particular stable isotope with respect to the naturally available proportion. As an example, Rhenium appears naturally in two stable forms, Rhenium-185 and Rhenium-187, being the proportion 37.4% to 62.6%. Enriched Rhenium-185 would be Rhenium with a proportion for example of 95% Rhenium-185 to 5% Rhenium-187. The activation rate (for example, the rate of creation of radioactive isotopes) depends on the abundance of the target isotopes, their cross section, their atomic weight, the half-life of the resulting radioactive isotopes, their concentration, the flux of activating particles or photons, and the activation time (for example, how long the activation process took place, ideally between 1 and 100 min).

Protons used to activate highly pure (i.e., 99.99% or higher) and enriched Iodine-124 can activate traces of other isotopes of iodine, as well as atoms in patient tissues. Moreover, protons interact with tissue as it is exploited in proton therapy. Activation protons would accordingly deposit a non-zero radiation dose (for example, energy deposited per unit of mass) on the tissue surrounding the tumor bed. The same applies to ions used for ion activation.

As for photons used for activation by means of photodisintegration, they also can deposit dose on their own, on patient tissue in the surrounding of the tumor bed.

In general, the above-mentioned examples prove that the proposed therapy may need detailed dosimetry calculations, such that the activation process of the target material in the medical device does not end up resulting in side effects or a radiation burden that overshadow the desired benefit of providing the irradiation of the tumor bed.

For dosimetry calculations, anatomical medical imaging modalities such as CT (X-ray computer tomography), MRI (magnetic resonance imaging) or US (ultrasound) can be used to determine the position of the implanted marker and the surrounding tissues. Based on that and the selected activation process, tissues potentially being exposed to activation neutrons, protons, ions, or photons can be defined. Monte Carlo simulations can be then used to obtain expected damage to the tumor bed and surrounding healthy tissue. An example of the proposed system comprises a software for planning the therapy and deriving the dose to the tumor bed, as well as the dose of surrounding healthy tissue that takes as input the position of the marker with respect to the anatomy of the patient as obtained by CT, MRI or US. In this case, we speak of pre-activation dosimetry.

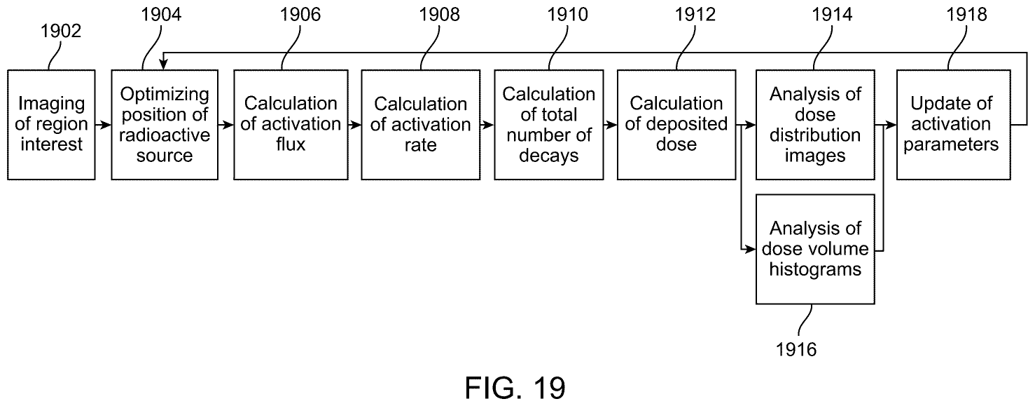

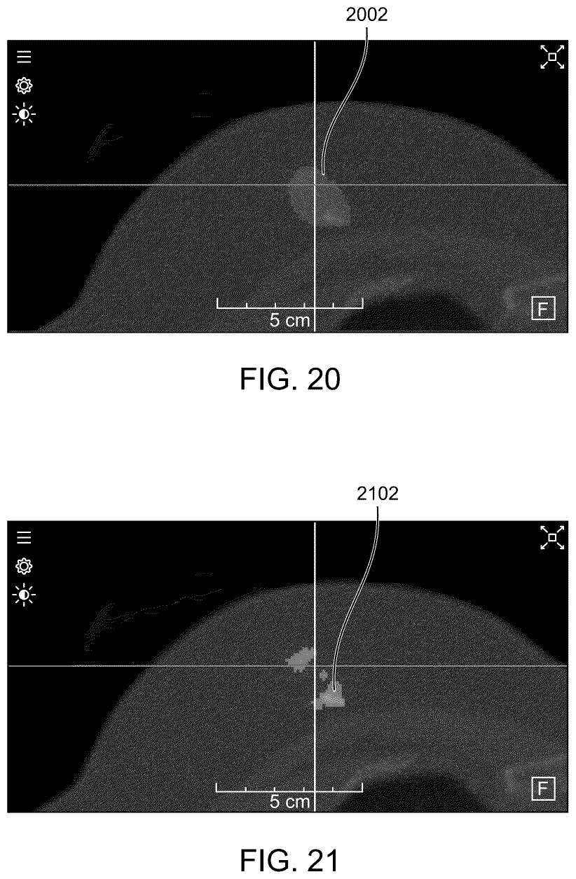

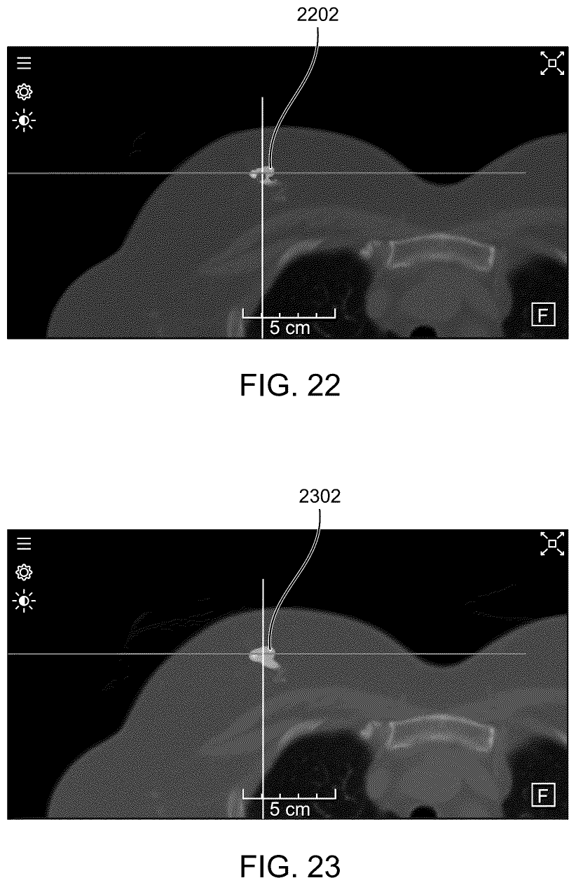

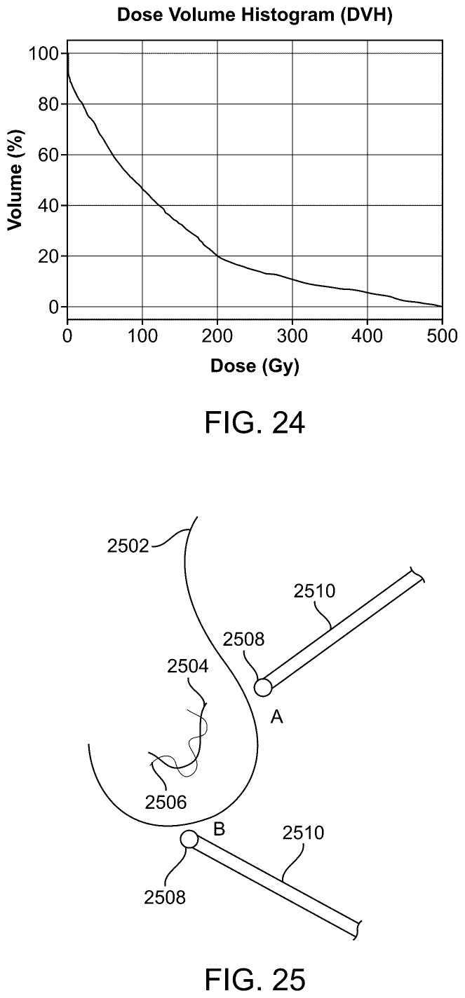

FIG. 19 shows an example of steps performed for pre-activation dosimetry for a single isotope, single radioactive source position, post-closure activation. The steps are:
a. Imaging the region of interest 1902 to figure out the exact position of the marker (e.g., using CT) which may include:
   i. Defining the tumor volume in the imaging (as shown for example in FIG. 20).
   ii. Segmenting the marker in the imaging (as shown for example in FIG. 21).
b. Optimizing the position of the radioactive source 1904 with respect to the position of the marker and the anatomy of the patient (since practically infinite options for positioning the radioactive source are available, optimization is needed to select the one that improves a particular criterion; a concrete example of such a criterion could be to obtain a rather homogeneous activation along the marker geometry)
c. Calculate the activation flux 1906 (e.g. neutron flux) of each section of the marker.
d. Calculate the activation rate 1908, e.g. the resulting radioactivity of each section of the marker given an activation time.
e. Calculate the total number of radioactive decays 1910 resulting from the activation for each section of the marker (as shown for example in FIG. 22).
f. Calculate the deposited dose 1912 given the total number of decays both in the tumor bed and in surrounding tissue (as shown for example in FIG. 23).
g. Plotting the dose volume histogram 1916 for the tumor bed and surrounding tissue (as shown for example in FIG. 24 for the tumor bed dose), and/or analyzing the dose distribution images 1914.
h. Updating activation parameters 1918 such as modifying the position of the radioactive source and activation time to obtain a desired dose to the tumor bed and/or the surrounding tissue.

The steps above, in particular, steps starting from step c, may also include calculating the activation of isotopes different than the stable isotope of the marker, such as those contained in human tissue to obtain a more precise dose distribution.

FIG. 20 shows an axial CT view of a breast cancer patient with an overlay of the tumor bed 2002 as a lighter grey area over the darker grey breast tissue. This image depicts the tumor bed which is desired to be treated.

FIG. 21 shows the same patient as in FIG. 20, now with an overlay showing the marker 2102 along the same axial CT view. In this case, since the marker is radiopaque and can be segmented from soft tissue based on the different Hounsfield units of the CT acquisition. This is depicted here by displaying the marker in lighter grey than the breast tissue. Here, the marker has a Rhenium-185 coating.

FIG. 22 shows the resulting radioactive distribution 2202 of Rhenium-186 using as a source a portable neutron source of Californium-252 placed on the skin of the patient close to the marker in the same patient as FIG. 20, for example, and in the same axial view, for 10 minutes. The radioactive distribution is given by a 3D distribution in Becquerel units, which stand for radioactive decays per second. The radioactive distribution is a continuous distribution depicted here in tones of grey.

FIG. 23 shows the dose distribution 2302 resulting from the decay of Rhenium-186 in the same patient example as FIG. 20 and along the same axial view. The dose distribution is a 3D distribution in Gray units, which stands for Joules per kilogram. The dose distribution is a continuous distribution depicted here in tones of grey.

FIG. 24 shows the dose volume histogram for the same patient example as FIG. 20 above. Here, the dose is provided by Rhenium-186 decay after activation with Californium-252 for about 10 minutes. The x-axis shows the deposited dose in the unit Gray (which is equivalent to Joule per kilogram) and the y-axis gives the percentage of tissue in the tumor bed that gets at least the value of the x-axis. Ideally, for example in breast cancer treatment a large percentage of the tumor bed gets a dose above 50 Gray. In this example, more than 60% of the tumor bed gets the desired dose. Such curves are used for planning and determine how activation should take place.

Post-activation dosimetry, i.e., dosimetry calculation after activation, may also be of use in order to calculate the real dose distribution due to the emissions of the radioactive marker (see option to use high energy gamma or positron emitting isotopes for imaging above).

To summarize aspects of the examples disclosed herein, an implantable marker may have any of the following characteristics, specifically a coating, or a radioisotope that is integral with the marker and forms at least a portion of the marker, which contains at least one stable isotope which can be activated by various activation means including:
Neutron activation
Proton activation
Ion activation
Photodisintegration
   With activation resulting in at least one radioactive isotope emitting either:
Very low-energy photons (X-rays and/or gamma rays),
Auger electrons,
Beta minus particles (ideally <3 MeV), or
Alpha particles (ideally <50 MeV)
   This yielding a majority of the emitted energy is deposited at a range <3 cm depth, and that the half-life of the resulting radioactive isotope is in the range of treatment isotopes in nuclear medicine/radiation therapy, such as, below 2 months.

The possibility of using a plurality of stable isotopes that can be activated in different spatial patterns along the marker provides degrees of freedom to modulate the spatial activation pattern and ultimately adapting the dose distribution to provide a proper tumor bed dose/healthy tissue dose ratio, or spare radiosensitive organs of a dose that could result in side-effects.

It should be considered that a coating is not a mandatory condition for any of the examples of the marker. The stable isotope may be part of the marker itself and thus the radioisotope forms at least a portion of the marker and is integral with the marker. One example of the marker is polypropylene with a Barium Sulfate (BaSO₄), Bismuth Sub carbonate, Bismuth Oxychloride or Bismuth Tri-Oxide as an additive for radiopacity. BaSO₄ may be replaced for example by Rhenium (IV) sulfide (ReS₂) or Rhenium (VII) sulfide (Re₂S₇), both of which are radiopaque and at the same time can be activated as they can be made of stable Rhenium-185 or stable Rhenium-187 or a mixture of both which can be activated with neutrons. Similarly, Holmiumoxide, Ho₂O₃, shows strong radiopacity and can be also activated by neutrons.

Similarly, if a ferromagnetic or superparamagnetic character is desired in order to increase visibility in MRI, the stable isotope may be for example particles of Yttrium and Holmium which show strong paramagnetic character. Both Yttrium and Holmium (e.g., Yttrium-89 and Holmium-165) can be activated by neutrons.

If the option of placing the stable isotope as part of the coating is desired, there are a variety of ways to bind the stable isotope to chemical molecules and complexes that can be used for coating. For example, stable Yttrium-89 or stable Lutetium-175 can be trapped in their ionic form using chelators. Both Yttrium-89 and Lutetium-175 can be activated by neutrons. It should also be mentioned that the coating can be multi-layer and multi-function. For example, hydrogel can be used as a binding layer. It should also be considered that the radiopaque, ferromagnetic, or superparamagnetic portion of the marker can be the coating and the stable isotope can be compounded with the base polymer such as polypropylene which is a permanent, non-absorbable. Other base polymers can be considered such as Nylon, silk, polyamide, Polyester, polyethylene. Base polymer can be absorbable such as polygalactin. We can consider having monofilament or multifilament as well. One can imagine having a stable isotope having a single filament that is woven in with a radiopaque, ferromagnetic, or superparamagnetic filament to create multifilament. The marker can also be made from metal and coated with a stable isotope. Metals can be such as nitinol, stainless steel, gold. Although the marker is described as elongated flexible, it can be short, like a bead. Multiple filaments may be combined to form a thread or multi-filament bundle, the filament may be absorbable in the body or non-resorbable. Other radiopaque or ferromagnetic or superparamagnetic fillers other than barium sulfate are well known in the art such as titanium dioxide, dense metals like gold, platinum, tantalum, etc., It may be noted that most stable isotopes mentioned in Tables 1, 2 and 3 have a large cross-section for X-rays used in radiography or CT and can be used both as target isotope for treatment as well as for making the marker radiopaque. As for superparamagnetism, both Yttrium and Holmium, in particular if combined in particles show strong paramagnetic behavior.

In additional detail, a source capable of emitting the required particles for the four activation means, namely placed either after the marker has been implanted (post closure activation) or inside the human body for a period of time of less than 1 hour before the surgical wound is closed (pre closure activation) may one or more of:
neutrons (for neutron activation).
protons (for positron activation).
ions (for ion activation).
high-energy photons or gamma rays (for photodisintegration).

The source mentioned above can be placed at different positions (and in some cases orientations) relative to the patient, as well as, for different durations in order to further modulate the spatial activation pattern and ultimately modulating the dose distribution.

In an alternative example at least one of the resulting radioactive isotopes also emits very low energy (<50 keV) to middle energy gamma rays (200-400 keV), such that the marker can be imaged using a gamma camera or a single photon emission computed tomography (SPECT).

In an alternative example at least one of the resulting radioactive isotopes also emits beta plus particles, also known as positrons, such that the marker can be imaged using a positron emission computed tomography (PET).

In an alternative example one of the resulting radioactive isotopes emits beta or alpha particles, such that the marker can be imaged using the resulting Bremsstrahlung using a gamma camera or a single photon emission computed tomography (SPECT).

In this specification, the definition of very low energy photons (X-rays or gamma rays) is when their energy is below 50 keV. For low energy photons (X-rays or gamma rays), the energy range is from 50 keV to 200 keV, while middle energy photons (X-rays or gamma rays) are when the energy range is from 200 keV to 400 keV. High energy photons (X-rays or gamma rays) are understood here to be from energies above 400 keV.

For a room-bound activation source the examples may be used:
Neutron beam from a neutron source for neutron activation.
Proton beam from a cyclotron or a proton beam therapy device for proton activation.
Ion beam for an ion therapy device for ion activation.
High energy photon beam from a Megavolt linear accelerator for photodisintegration.
High energy gamma ray beam from a radioactive isotope-based irradiation unit for Photodisintegration.
A high energy proton beam from a Megavolt Linear accelerator directed to an X-ray target using an electron beam guide for producing X-rays that on their own enable photodisintegration.

For portable activation, examples of activators may include a portable applicator with a radioactive source comprising:
A neutron source using a radioactive material spontaneously emitting neutrons or a combination of radioactive isotopes that result in a net spontaneous emission of Neutrons for neutron activation (e.g. AmBe or Californium-252).
A proton source using a radioactive material spontaneously emitting protons or a combination of radioactive isotopes that result in a net spontaneous emission of protons for proton activation (e.g. Lutetium-151 or Thalium-147).
An ion source using a radioactive material spontaneously emitting ions or a combination of radioactive isotopes that result in a net spontaneous emission of ions for ion activation (e.g. Actinium-225 or Radium-223).
A high energy photon source resulting from irradiation of a material with a Megavolt linear electron beam for photodisintegration.
A gamma ray source using a radioactive material spontaneously emitting gamma rays or a combination of radioactive isotopes that result in a net spontaneous emission of gamma rays for photodisintegration (e.g. Cobalt-60).

Since not only the stable isotope in the marker can be activated, or since the activation process itself can deposit a dose in tissue, an example of the marker also may comprise software for planning the therapy and deriving the dose to the marker, as well as the dose of surrounding healthy tissue. This software can be used prior to the therapy for planning, but also after the therapy to evaluate the real dose distribution.

In a case of a tissue biopsy, a clip is typically left in the location of biopsy to mark the area of tissue removal. As seen and described in FIG. 11, clips can be coated with therapeutic coatings or formed at least partially from a therapeutic agent. These clips can provide therapies as mentioned earlier such as anti-microbial, anti-inflammatory, anesthetic/pain relief and others mentioned.

If the pathology comes back malignant for example, it is feasible that if the tumor is small enough the clip which was placed in the suspected region can then be activated (externally as in FIGS. 12, 13, 16 and 17, or using a needle as in FIG. 14 or an endoscopic approach as in FIG. 15) if the clip had a radioactive activated coating described earlier or is formed from a radioactive activated material. So, if the tissue does not require further treatment, the stable isotope is passive and has no effect on the patient. However, if therapy is required, a radioactive source is used to activate the clip thus providing radiation.

The disclosure above describes various therapeutic coatings as well as radioactive coatings. In other examples, ablative coatings may be used with the marker. For example, metal coatings that can be activated externally. Gold nanoparticles can be activated and generate heat by IR (infrared) light for example or RF (radiofrequency)/microwave energy. Magnetic particles can be activated using a magnetic field. For example, gold, iron oxide nanoparticles, magnetite and mag-hemite can be used to be activated by external magnetic field. Coatings are disclosed that are applied to markers that can generate thermal/ablative therapies when activated; either through optical (near IR) or magnetic.

Any of the features mentioned earlier can be combined in any permutation or combination. So, for example and anti-microbial coating can be combined with a radioactive coating, etc. be delivered to a target tissue, and any marker such as a clip or filament may be used with any therapeutic agent.

### Notes and Examples

The following, non-limiting examples, detail certain aspects of the present subject matter to solve the challenges and provide the benefits discussed herein, among others.

Example 1 an implantable marker configured to deliver a therapy to a patient, the implantable marker comprising: a marker configured to enhance visibility of a treatment region of the patient when implanted in the patient and viewed with an imaging modality; and a target isotope coupled to at least a portion of the marker, wherein the target isotope has a non-radioactive state and a radioactive state, wherein at least a portion of the target isotope is configured to be induced from the non-radioactive state to the radioactive state when the target isotope is irradiated with one or more of gamma rays, neutrons, alpha particles, beta particles, or ions; and wherein the target isotope emits a localized therapeutic dose of radiation to target tissue adjacent the implantable marker when the target isotope is in the radioactive state.

Example 2 is the implantable marker of Example 1, wherein the target isotope in the radioactive state emits one or more of alpha particles of less than or equal to 10 MeV, beta particles of less than or equal to 3 MeV, low energy gamma rays of less than or equal to 50 keV, or Auger electrons.

Example 3 is the implantable marker of any one of Examples 1-2, wherein the target isotope forms at least a portion of the marker.

Example 4 is the implantable marker of any one of Examples 1-3, wherein the target isotope is a coating disposed on the marker.

Example 5 is the implantable marker of any one of Examples 1-4, wherein the target isotope comprises a first target isotope and a second target isotope, wherein the first target isotope in the radioactive state emits radiation configured to penetrate the target tissue a first distance, and wherein the second target isotope in the radioactive state emits radiation configured to penetrate the target tissue a second distance different than the first distance.

Example 6 is the implantable marker of any one of Examples 1-5, wherein the implantable marker further comprises: a second marker configured to enhance visibility of the treatment region of the patient when implanted in the patient and viewed with a second imaging modality the same as the imaging modality or different therefrom; and a second a target isotope coupled to at least a portion of the second marker, wherein the second target isotope has a non-radioactive state and a radioactive state, and wherein at least a portion of the second target isotope is induced from the non-radioactive state to the radioactive state when the second target isotope is irradiated with one or more of gamma rays, neutrons, alpha particles, beta particles, or ions.

Example 7 is the implantable marker of any one of Examples 1-6, wherein the marker comprises an elongate flexible filament or a clip.

Example 8 is the implantable marker of any one of Examples 1-7, wherein the marker is radiopaque, echogenic, ferromagnetic, or superparamagnetic.

Example 9 is a system for marking tissue, comprising the implantable marker of any one of Examples 1-8; and a radioactive source configured to be disposed in proximity to the target isotope when the target isotope is in the non-radioactive state, and wherein the radioactive source is configured to emit a beam of radiation directed toward the target isotope that induces the isotope from the non-radioactive state to the radioactive state.

Example 10 is the system of Example 9, wherein the beam of radiation comprises one or more of high energy photons, beta particles, neutrons, alpha particles, or ions.

Example 11 is the system of any one of Examples 9-10, further comprising: a holder and a handle, the holder disposed at one end of the handle, and wherein the radioactive source is disposed in the holder; or a needle with a tip, wherein the radioactive source is disposed in the tip; or a cannula with a tip, wherein the radioactive source is disposed in the tip of the cannula.

Example 12 is a method for treating tissue, comprising: attaching an implantable marker to the tissue, wherein the implantable marker is configured to enhance visibility of a treatment region when implanted in a patient and viewed with an imaging modality, wherein the implantable marker comprises a target isotope, the target isotope having a non-radioactive state and a radioactive state, and wherein the attaching is performed when the target isotope is in the non-radioactive state; irradiating the target isotope with one or more of high-energy photons, beta particles, neutrons, alpha particles, or ions; inducing the target isotope from the non-radioactive state to the radioactive state; and irradiating the tissue with radiation emitted from the target isotope in the radioactive state, the radiation directed at the tissue and the radiation comprising one or more of alpha particles of less than 10 MeV, beta particles of less than 3 MeV, low-energy gamma rays of less than 50 keV, or Auger electrons.

Example 13 is the method of Example 12, wherein a surgical wound provides access to the tissue, and wherein the irradiating the target isotope is performed while the surgical wound is open, prior to closure of the surgical wound.

Example 14 is the method of any one of Examples 12-13, wherein a surgical wound provides access to the tissue, and wherein the irradiating the target isotope is performed after closure of the surgical wound.

Example 15 is the method of any one of Examples 12-14, wherein the irradiating the target isotope comprises disposing a radioactive activation source adjacent the target isotope, and wherein disposing the radioactive activation source is performed percutaneously, transluminally, or invasively by inserting the radioactive activation source in the patient.

Example 16 is the method of any one of Examples 12-15, wherein the radioactive activation source is configured to emit the one or more of high-energy photons, beta particles, neutrons, alpha particles, or ions.

Example 17 is the method of any one of Examples 12-16, wherein the irradiating the target isotope comprises moving a radioactive activation source relative to the implantable marker during the irradiation of the target isotope.

Example 18 is the method of any one of Examples 12-17, wherein the irradiating the target isotope comprises irradiating the target isotope with a beam of radiation, and changing a relative position and an orientation of the beam of radiation during irradiation of the target isotope.

Example 19 is the method of any one of Examples 12-18, further comprising calculating a deposited dose resulting from irradiating the radioactive isotope and inducing the radioactive isotope from the non-radioactive state to the radioactive state.

Example 20 is the method of any one of Examples 12-19, further comprising calculating a deposited dose resulting from irradiating the tissue and the implantable marker, and inducing the tissue or inducing the implantable marker, from a non-radioactive state to a radioactive state, and wherein the calculating excludes a deposited dose resulting from irradiating the radioactive isotope and inducing the radioactive isotope from the non-radioactive state to the radioactive state.

Example 21 is the method of any one of Examples 12-20, wherein the irradiating the target isotope comprising irradiating the target isotope with a radiation source, the method further comprising optimizing a deposited dose by defining a relative path of the radioactive source relative to the implantable marker, wherein the deposited dose results from inducing the radioactive isotope from the non-radioactive state to the radioactive state, or the deposited dose results from inducing the tissue or the implantable marker from a non-radioactive state to a radioactive state.

Example 22 is the method of any one of Examples 12-21, wherein the irradiating the target isotope comprises irradiating the target isotope with a beam of radiation from a radiation source, the method further comprising optimizing a deposited dose by defining a relative path of the beam of radiation relative to the implantable marker, wherein the deposited dose results from inducing the radioactive isotope from the non-radioactive state to the radioactive state, or the deposited dose results from inducing the tissue or the implantable marker from a non-radioactive state to a radioactive state.

Example 23 is the method of any one of Examples 12-22, wherein the implantable marker comprises an elongate flexible filament or a clip.

Example 24 is the method of any one of Examples 12-23, wherein the marker is radiopaque, echogenic, ferromagnetic, or superparamagnetic.

In Example 25, the apparatuses, systems or methods of any one or any combination of Examples 1-24 can optionally be configured such that all elements or options recited are available to use or select from.

The above detailed description includes references to the accompanying drawings, which form a part of the detailed description. The drawings show, by way of illustration, specific embodiments in which the invention can be practiced. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

1. An implantable marker configured to deliver a therapy to a patient, the implantable marker comprising:
a marker configured to enhance visibility of a treatment region of the patient when implanted in the patient and viewed with an imaging modality; and
a target isotope coupled to at least a portion of the marker, wherein the target isotope has a non-radioactive state and a radioactive state,
wherein at least a portion of the target isotope is configured to be induced from the non-radioactive state to the radioactive state when the target isotope is irradiated with one or more of gamma rays, neutrons, alpha particles, beta particles, or ions; and
wherein the target isotope emits a localized therapeutic dose of radiation to target tissue adjacent the implantable marker when the target isotope is in the radioactive state.

2. The implantable marker of claim 1, wherein the target isotope in the radioactive state emits one or more of alpha particles of less than or equal to 10 MeV, beta particles of less than or equal to 3 MeV, low energy gamma rays of less than or equal to 50 keV, or Auger electrons.

3. The implantable marker of claim 1, wherein the target isotope forms at least a portion of the marker.

4. The implantable marker of claim 1, wherein the target isotope is a coating disposed on the marker.

5. The implantable marker of claim 1, wherein the target isotope comprises a first target isotope and a second target isotope, wherein the first target isotope in the radioactive state emits radiation configured to penetrate the target tissue a first distance, and wherein the second target isotope in the radioactive state emits radiation configured to penetrate the target tissue a second distance different than the first distance.

6. The implantable marker of claim 1, wherein the implantable marker further comprises:
a second marker configured to enhance visibility of the treatment region of the patient when implanted in the patient and viewed with a second imaging modality the same as the imaging modality or different therefrom; and
a second a target isotope coupled to at least a portion of the second marker, wherein the second target isotope has a non-radioactive state and a radioactive state, and wherein at least a portion of the second target isotope is induced from the non-radioactive state to the radioactive state when the second target isotope is irradiated with one or more of gamma rays, neutrons, alpha particles, or beta particles.

7. The implantable marker of claim 1, wherein the marker comprises an elongate flexible filament or a clip.

8. The implantable marker of claim 1, wherein the marker is radiopaque, echogenic, ferromagnetic, or superparamagnetic.

9. A system for marking a tissue, comprising:
the implantable marker of claim 1; and
a radioactive source configured to be disposed in proximity to the target isotope when the target isotope is in the non-radioactive state, and wherein the radioactive source is configured to emit a beam of radiation directed toward the target isotope that induces the isotope from the non-radioactive state to the radioactive state.

10. The system of claim 9, wherein the beam of radiation comprises one or more of high energy photons, beta particles, neutrons, alpha particles, or ions.

11. The system of claim 9, further comprising:
a holder and a handle, the holder disposed at one end of the handle, and wherein the radioactive source is disposed in the holder; or
a needle with a tip, wherein the radioactive source is disposed in the tip; or
a cannula with a tip, wherein the radioactive source is disposed in the tip of the cannula.

12. A method for treating tissue, comprising:
attaching an implantable marker to the tissue, wherein the implantable marker is configured to enhance visibility of a treatment region when implanted in a patient and viewed with an imaging modality,
wherein the implantable marker comprises a target isotope, the target isotope having a non-radioactive state and a radioactive state, and wherein the attaching is performed when the target isotope is in the non-radioactive state;
irradiating the target isotope with one or more of high-energy photons, beta particles, neutrons, alpha particles, or ions;
inducing the target isotope from the non-radioactive state to the radioactive state; and
irradiating the tissue with radiation emitted from the target isotope in the radioactive state, the radiation directed at the tissue and the radiation comprising one or more of alpha particles of less than 10 MeV, beta particles of less than 3 MeV, low-energy gamma rays of less than 50 keV, or Auger electrons.

13. The method of claim 12, wherein a surgical wound provides access to the tissue, and wherein the irradiating the target isotope is performed while the surgical wound is open, prior to closure of the surgical wound.

14. The method of claim 12, wherein a surgical wound provides access to the tissue, and wherein the irradiating the target isotope is performed after closure of the surgical wound.

15. The method of claim 12, wherein the irradiating the target isotope comprises disposing a radioactive activation source adjacent the target isotope, and wherein disposing the radioactive activation source is performed percutaneously, transluminally, or invasively by inserting the radioactive activation source in the patient.

16. The method of claim 15, wherein the radioactive activation source is configured to emit the one or more of high-energy photons, beta particles, neutrons, alpha particles, or ions.

17. The method of claim 12, wherein the irradiating the target isotope comprises moving a radioactive activation source relative to the implantable marker during the irradiation of the target isotope.

18. The method of claim 12, wherein the irradiating the target isotope comprises irradiating the target isotope with a beam of radiation, and changing a relative position and an orientation of the beam of radiation during irradiation of the target isotope.

19. The method of claim 12, further comprising calculating a deposited dose resulting from irradiating the radioactive isotope and inducing the radioactive isotope from the non-radioactive state to the radioactive state.

20. The method of claim 12, further comprising calculating a deposited dose resulting from irradiating the tissue and the implantable marker, and inducing the tissue or inducing the implantable marker, from a non-radioactive state to a radioactive state, and wherein the calculating excludes a deposited dose resulting from irradiating the radioactive isotope and inducing the radioactive isotope from the non-radioactive state to the radioactive state.

21. The method of claim 12, wherein the irradiating the target isotope comprising irradiating the target isotope with a radiation source, the method further comprising optimizing a deposited dose by defining a relative path of the radioactive source relative to the implantable marker, wherein the deposited dose results from inducing the radioactive isotope from the non-radioactive state to the radioactive state, or the deposited dose results from inducing the tissue or the implantable marker from a non-radioactive state to a radioactive state.

22. The method of claim 12, wherein the irradiating the target isotope comprises irradiating the target isotope with a beam of radiation from a radiation source, the method further comprising optimizing a deposited dose by defining a relative path of the beam of radiation relative to the implantable marker, wherein the deposited dose results from inducing the radioactive isotope from the non-radioactive state to the radioactive state, or the deposited dose results from inducing the tissue or the implantable marker from a non-radioactive state to a radioactive state.

23. The method of claim 12, wherein the implantable marker comprises an elongate flexible filament or a clip.

24. The method of claim 12, wherein the marker is radiopaque, echogenic, ferromagnetic, or superparamagnetic.
